(19) United States
(12) Patent Application Publication
(10) Pub. No.: US 2020/0281531 A1
(43) Pub. Date: Sep. 10, 2020

(54) ELECTROMEDICAL APPARATUS, A METHOD FOR MANUFACTURING THE ELECTROMEDICAL APPARATUS AND A METHOD FOR DEPLOYING THE ELECTROMEDICAL APPARATUS

(71) Applicant: City University of Hong Kong, Kowloon (HK)

(72) Inventors: Stella W. Pang, Kowloon (HK); Yuanhao Xu, Kowloon (HK)

(21) Appl. No.: 16/295,211

(22) Filed: Mar. 7, 2019

(51) Int. Cl. A61B 5/00; A61B 5/04; B32B 7/12; B32B 27/08; B32B 27/28; B32B 37/12; B32B 37/00

(52) U.S. Cl. CPC A61B 5/6885; A61B 5/04001; B32B 7/12; B32B 27/08; B32B 27/283; B32B 27/281; A61N 1/0551; B32B 37/025; A61B 2562/0209; A61B 2562/043; A61B 2562/125; A61B 2562/168; A61B 2562/028; B32B 37/12

(57) ABSTRACT

An electromedical apparatus, a method for manufacturing the electromedical apparatus and a method for deploying the electromedical apparatus. The electromedical apparatus includes an electrical component arranged to facilitate an electrical signal transmission between an electrical device and a target; and a mechanical component physically connected to the electrical component; wherein the mechanical component includes a fluidic structure arranged to modify at least one physical property of the mechanical component, so as to facilitate a deployment of the electromedical apparatus on the target.

### TECHNICAL FIELD

The present invention relates to an electromedical apparatus, a method for manufacturing the electromedical apparatus and a method for deploying the electromedical apparatus, and particularly, although not exclusively, to an electromedical apparatus having a controllable mechanical structure.

### BACKGROUND

Electromedical apparatus may be used to record electrical activity of a biological target. For example, electrocardiography may record the activity of the heart of a living species over a period of time, in which gel pads may be used to attach electrodes over the skin near near the position of the heart to detect the electrical changes on the skin that represent heartbeats.

Neural activities may also be recorded using electromedical apparatus, however the size of the electrodes of such apparatus designed for neural applications are much smaller when compared to electrocardiography, so as to precisely record activities of cells and tissues. These micro- or nano-scale electrodes may also be used in stimulating the cells in diagnostic tests or medical treatments.

### SUMMARY OF THE INVENTION

In accordance with a first aspect of the present invention, there is provided an electromedical apparatus comprising: an electrical component arranged to facilitate an electrical signal transmission between an electrical device and a target; and a mechanical component physically connected to the electrical component; wherein the mechanical component includes a fluidic structure arranged to modify at least one physical property of the mechanical component, so as to facilitate a deployment of the electromedical apparatus on the target.

In an embodiment of the first aspect, the target is a biological target.

In an embodiment of the first aspect, the fluidic structure comprises at least one fluidic channel arranged to at least temporary accommodate a fluid therein.

In an embodiment of the first aspect, the mechanical component comprises a multi-layered structure including at least a first layer of material and a second layer of material.

In an embodiment of the first aspect, the at least one fluidic channel is defined by at least one trench formed on the second layer of material and encapsulated by the first layer of material.

In an embodiment of the first aspect, the first layer of material and the second layer of material are biocompatible, and are provided with desired mechanical and chemical properties, e.g. being mechanically flexible, electrically insulating, etc.

In an embodiment of the first aspect, the first layer of material includes polyimide.

In an embodiment of the first aspect, the second layer of material includes polydimethylsiloxane.

In an embodiment of the first aspect, the electrical component includes at least one metal electrode arranged on the first layer of material or the second layer of material.

In an embodiment of the first aspect, the at least one physical property includes a mechanical stiffness of the mechanical component, and wherein the mechanical stiffness is modified in response to a variation of an internal pressure of the fluidic channel.

In an embodiment of the first aspect, when the electrical apparatus operates in a first condition, the mechanical stiffness of the mechanical component is higher than that of the mechanical component when the electrical apparatus operates in a second condition.

In an embodiment of the first aspect, the mechanical component is arranged to prevent bending or buckling of the electromedical apparatus during the deployment of the electromedical apparatus on the target.

In an embodiment of the first aspect, the electrical component and the mechanical component combine to define an elongated body, and wherein the fluidic channel runs through at least a portion of the length of the elongated body.

In an embodiment of the first aspect, the fluidic structure further comprises an inlet and an outlet both in connection with the fluidic channel.

In an embodiment of the first aspect, the inlet and the outlet are defined at a first end of the elongated body.

In an embodiment of the first aspect, the electromedical apparatus further comprises a tapered tip defined at a second end of the elongated body.

In an embodiment of the first aspect, the fluidic channel comprises a U-shaped portion or a fluid returning portion of different shapes or designs at or proximate to the second end of the elongated body.

In an embodiment of the first aspect, the electrical component and the mechanical component combine to operate as a neural probe.

In an embodiment of the first aspect, the fluidic structure further comprises at least one fluidic cavity in fluid communication with a respective fluidic channel.

In an embodiment of the first aspect, the at least one physical property includes a physical dimension of each of the at least one fluidic cavity, and wherein the physical dimension is modified in response to a variation of an internal pressure in the fluidic channel and/or the fluidic cavity.

In an embodiment of the first aspect, in response to the variation of the internal pressure in the fluidic cavity, the at least one fluidic cavity is arranged to move a respective portion of the electrical component with respect to the target during the deployment of the electromedical apparatus on the target.

In an embodiment of the first aspect, the mechanical component is arranged to improve a physical contact between the electrical component and the target.

In an embodiment of the first aspect, the electrical component comprises an array of electrodes arranged to electrically contact with the target.

In an embodiment of the first aspect, the fluidic structure comprises a plurality of fluidic cavities, wherein the plurality of fluidic cavities defines an array of fluidic cavities.

In an embodiment of the first aspect, the size and dimension of the array of electrodes are different from those of the array of fluidic cavities.

In an embodiment of the first aspect, each of the electrodes in the electrical component aligns with a corresponding fluidic cavity in the mechanical component.

In an embodiment of the first aspect, the electrical component further comprises a counter electrode adjacent to each electrode in the array of electrodes.

In an embodiment of the first aspect, each electrode and counter electrode pair is arranged to estimate a position of the electrode with respect to the target so as to evaluate the physical contact between the electrical component and the target.

In an embodiment of the first aspect, the electrical component and the mechanical component combine to operate as a micro-electrode array apparatus.

In an embodiment of the first aspect, the electromedical apparatus further comprises a pump arranged to manipulate a flow of fluid through the fluidic channel under a predetermine pressure.

In accordance with a second aspect of the present invention, there is provided a method for deploying the electromedical apparatus in accordance with the first aspect on a target, comprising the steps of: increasing the internal pressure of the fluidic channel; implanting the neural probe in the target; and decreasing the internal pressure of the fluidic channel after a disposition of the electrical component in a first predetermined position.

In an embodiment of the second aspect, the method further comprises the step of controlling the internal pressure of the fluidic channel so as to move the electrical component from the first predetermined position to a second predetermined position.

In accordance with a third aspect of the present invention, there is provided a method for deploying the electromedical apparatus in accordance with the first aspect on a target, comprising the steps of: disposing the micro-electrode array apparatus on the target; an controlling the internal pressure of each of the plurality of fluidic cavities so as to improve the physical contact between the electrical component and the target at each respective portion of the electrical component.

In an embodiment of the third aspect, the method further comprises the step of estimating a position of each of the electrodes with respect to the target to evaluate the physical contact between each of the electrodes and the target.

In accordance with a fourth aspect of the present invention, there is provided a method for manufacturing an electromedical apparatus, comprising the steps of: defining a first layer of material with a fluidic structure; and combining the first layer of material with a second layer of material to form a multi-layer structure defining a mechanical component with the fluidic structure; and providing an electrical component to physically connect with the mechanical component, the electrical component is arranged to facilitate an electrical signal transmission between an electrical device and a target; wherein the fluidic structure is arranged to modify at least one physical property of the mechanical component, so as to facilitate a deployment of the electromedical apparatus on the target.

In an embodiment of the fourth aspect, the step of combining the first layer of material with a second layer of material comprises the step of stacking and binding the first layer of material onto a second layer of material.

In an embodiment of the fourth aspect, the first layer of material and the second layer of material are combined via chemical bondings.

In an embodiment of the fourth aspect, the first layer of material and the second layer of material are combined via a layer of adhesive material.

In an embodiment of the fourth aspect, the mechanical component is formed using a reversal imprint process.

In an embodiment of the fourth aspect, the step of defining a first layer of material with a fluidic structure comprises the step of applying the first layer of material on a stamp so as to construct the fluidic structure on the first layer of material.

In an embodiment of the fourth aspect, the step of defining a first layer of material with a fluidic structure comprises the step of coating a first anti-sticking layer on the stamp to modify a surface energy prior to applying the first layer of material on the stamp.

In an embodiment of the fourth aspect, the method further comprises the step of: transferring the first layer of material from the stamp to a handling substrate;

and releasing the first layer of material from the handling substrate after binding the first layer of material onto the second layer of material.

In an embodiment of the fourth aspect, the step of transferring the first layer of material from the stamp to a handling substrate comprises the steps of: coating a second anti-sticking layer on the handling substrate; stacking the first layer of material apply on the stamp to the handling substrate; and releasing the first layer of material from the stamp.

In an embodiment of the fourth aspect, the first or the second anti-sticking layer comprise FOTS and/or MOPTS, or other coating materials to provide the desired surface energy

In an embodiment of the fourth aspect, the step of providing the electrical component comprises the step of fabricating at least one electrode on the first and/or second layer of material.

In an embodiment of the fourth aspect, the step of fabricating the at least one electrode on the first and/or the second layer of material comprises the steps of: depositing a metal layer on the first layer of material and/or the second layer of material; and patterning the metal layer by photolighography and etching of the metal layer.

In an embodiment of the fourth aspect, the at least one electrode is fabricated prior to the step of combining the first layer of material with the second layer of material.

In an embodiment of the fourth aspect, the method further comprises the step of covering the at least one electrode with a passivation layer prior to combining the first layer of material with the second layer material.

In an embodiment of the fourth aspect, the method further comprises the step of exposing at least one electrical contact of the electrical component by etching.

In an embodiment of the fourth aspect, the at least one electrode comprises Au and/or Cr.

In an embodiment of the fourth aspect, the first layer of material and the second layer of material are biocompatible, and are provided with desired mechanical and chemical properties, e.g. being mechanically flexible, electrically insulating, etc.

In an embodiment of the fourth aspect, the first layer of material includes polyimide.

In an embodiment of the fourth aspect, the second layer of material includes polydimethylsiloxane.

In an embodiment of the fourth aspect, the first layer of material and the second layer of material are combine via a layer of binding material sandwiched between the first and the second layers of material.

In an embodiment of the fourth aspect, the binding material includes polymethyl methacrylate.

In an embodiment of the fourth aspect, the electromedical apparatus operates as a neural probe.

In an embodiment of the fourth aspect, the electromedical apparatus operates as a micro-electrode array apparatus.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of example, with reference to the accompanying drawings in which:

FIGS. 1A and 1B are schematic diagraphs of an electromedical apparatus in accordance with embodiments of the present invention;

FIG. 2A is a cross-sectional view of the electromedical apparatus of FIG. 1A;

FIG. 2B is a cross-sectional view of a single electrode unit of the electromedical apparatus of FIG. 1B;

FIGS. 2C and 2D are cross-sectional view of two embodiments of the electromedical apparatus of FIG. 1B;

FIG. 3 is an illustration showing a process for fabricating of an imprinting stamp for use in fabricating the electromedical apparatus in accordance with an embodiment of the present invention;

FIGS. 4A, 4B and 4C are illustrations showing a process flow for fabricating the electromedical apparatus of FIG. 1A;

FIGS. 5A, 5B, and 5C are illustrations showing a process flow for fabricating the electromedical apparatus of FIG. 1B;

FIG. 6 shows microscopic images showing the electrical component and the mechanical component of the electromedical apparatus of FIG. 1A;

FIG. 7 is an image showing a controller board in connection with the electromedical apparatus in accordance with embodiments of the present invention;

FIG. 8 is a microscopic image showing the mechanical component of the electromedical apparatus of FIG. 1A;

FIGS. 9A and 9B are illustrations showing the parameters of the electromedical apparatus of FIG. 1A in an experiment for evaluating the buckling performance of the electromedical apparatus;

FIGS. 9C and 9D are distribution graph and plot showing the results of the experiment according to FIGS. 9A and 9B;

FIGS. 10A, 10B, 10C, 10D, and 10E are plots showing the relationships between critical buckling force vs internal fluidic pressure applied to the electromedical apparatus of FIG. 1A with different design parameters;

FIGS. 11A, 11B, and 11C are plots showing measured and simulated critical buckling force at different fluidic pressure applied to the electromedical apparatus of FIG. 1A with different design parameters;

FIGS. 12A, 12B, and 12C are plots showing measured critical buckling force at different fluidic pressure applied to the electromedical apparatus of FIG. 1A with different design parameters;

FIG. 13A is an illustration showing a setup for gel movement test for the electromedical apparatus of FIG. 1A;

FIG. 13B is a plot showing a relationship between the insertion force in the gel at different fluidic pressure applied to the electromedical apparatus of FIG. 13A;

FIGS. 13C and 13D are micrographs and plots showing the displacement of the electromedical apparatus of FIG. 13A and comparison metal probes;

FIGS. 14A and 14B are photographic images showing an insertion of the electromedical apparatus of FIG. 1A in a rat brain without and with an applied fluidic pressure in the fluidic channel;

FIG. 15A is an illustration showing an adjustment of the position of the electrode in the electromedical apparatus of FIG. 1B towards the target of a retina surface with applied fluidic pressure;

FIGS. 15B, 15C and 15D are a photographic image, a schematic diagram and a microscopic image showing the electromedical apparatus of FIG. 1B;

FIG. 16 is an image showing an implanted MEA in accordance with an embodiment of the present invention on a rat eye; and

FIGS. 17A, 17B and 17C are plots showing the record signals in a stimulation test of the electromedical apparatus of FIG. 1B on a biological target with different stimulation current pulses.

### DETAILED DESCRIPTION OF THE PREFERRED EMBODIMENT

The inventors have, through their own research, trials and experiments, devised that, electromedical apparatus, such as implantable neural probe(s) may be used to record and stimulate neural activities. For example, neural probes may be implanted into different organs or tissues (e.g. brain, eye, and ear). The neural probes include a biocompatibility for many neural probe applications, especially for those that require long term usage.

In some example embodiments, biocompatible polymer-based neural probes may be used for neural prosthetics. Preferably, different biocompatible polymers such as polydimethylsiloxane (PDMS), parylene-C, liquid crystal polymer (LCP), SU-8, and polyimide (PI) may be used to fabricate flexible neural probes to reduce human body responses after implantation.

However, these flexible polymer-based neural probes are relatively soft and therefore may cause difficulties in inserting into the targeted tissues. For example, due to the high flexibility of PDMS, these neural probes were difficult to be inserted into the brain or some other targeted tissues, and therefore may be more suitable to be used in surface neural prostheses applications.

In addition, the flexibility of the neural probes may also induce a certain degree of tissue damage during brain micro motions after the probes are implanted. Preferably, neural probes with higher flexibility may cause less tissue damage. However, a minimum probe stiffness may be needed to continue implant into the brain tissue.

In one example embodiment, flexible probe insertion may be accomplished using a probe stiffener, in which the flexible probe may be attached to a rigid silicon (Si) substrate with a biodegradable glue such as polyethylene glycol (PEG). Alternatively, biodissolvable materials such as silk polymer may be coated onto a surface of the neural probes to temporarily enhance neural probe stiffness during probe insertion.

In the abovementioned examples, the flexible probes are made more rigid during probe insertion, and then the probes are maintained to be highly flexible after the successful probe insertion. The process is irreversible.

In accordance with an embodiment of the present invention, the flexibility or the rigidity of the probes may be dynamically controlled. For example, the probes are provided with a fluidic channel in the probe body such that the flexibility of the neural probe may be dynamically controlled by manipulating the fluidic pressure in embedded channels.

Preferably, the neural probes may be controlled to be rigid during insertion, and then returned to inherent flexibility after the insertion. The neural probe flexibility may be adjusted by the using different probe materials, dimensions, and fluidic pressure in the embedded microchannels.

For example, neural probes may be fabricated in PDMS, PI, and/or PDMS/PI hybrid substrates. The polymer-based neural probes are biocompatible and their mechanical properties can be optimized. Preferably, PDMS and PI polymers have different Young's modulus, and therefore they can provide different degrees of stiffness.

Without wishing to be bound by theory, PDMS may be too soft for neural probe implantation into the brain tissue. PI is more rigid compared to PDMS. Accordingly, a combination of PDMS and PI layers in neural probes may be tuned to reach the appropriate flexibility. For neural probe implantation, the fluidic pressure in the embedded microchannels of PDMS/PI neural probes may be adjusted to provide a desired stiffness. The fluidic pressure in the microchannels could then be released after implantation so that the neural probes will be highly flexible for low tissue damage.

The inventors devised that neural probes in PDMS may be too flexible to be implanted into brain tissue, even with fluidic pressure in the embedded channels. On the other hand, the minimum critical buckling force for the PI probes was found to be 3.0 mN for 200 μm wide probes with 50 μm wide channels, which may be higher than the required force of 0.4-1.0 mN to penetrate into the brain tissue.

Without wishing to be bound by theory, hybrid PDMS/PI neural probes with the same dimensions and fluidic pressure may be more flexible with lower critical buckling force of 1.1 mN and rigid enough to penetrate the brain tissue. The flexibility of the PDMS/PI neural probes may be optimized at 0.25-1.25 mN depending on the fluidic pressure in the microchannel.

In one example embodiment, as fabricated neural probes were tested and inserted into a 0.6% agarose gel that mimicked the softness of the brain tissue. As the fluidic pressure was increased from 0 to 60 kPa, these probes were able to penetrate into the gel. After insertion, releasing the fluidic pressure allowed the probe to become more flexible to avoid tissue damage for chronic recording and stimulation.

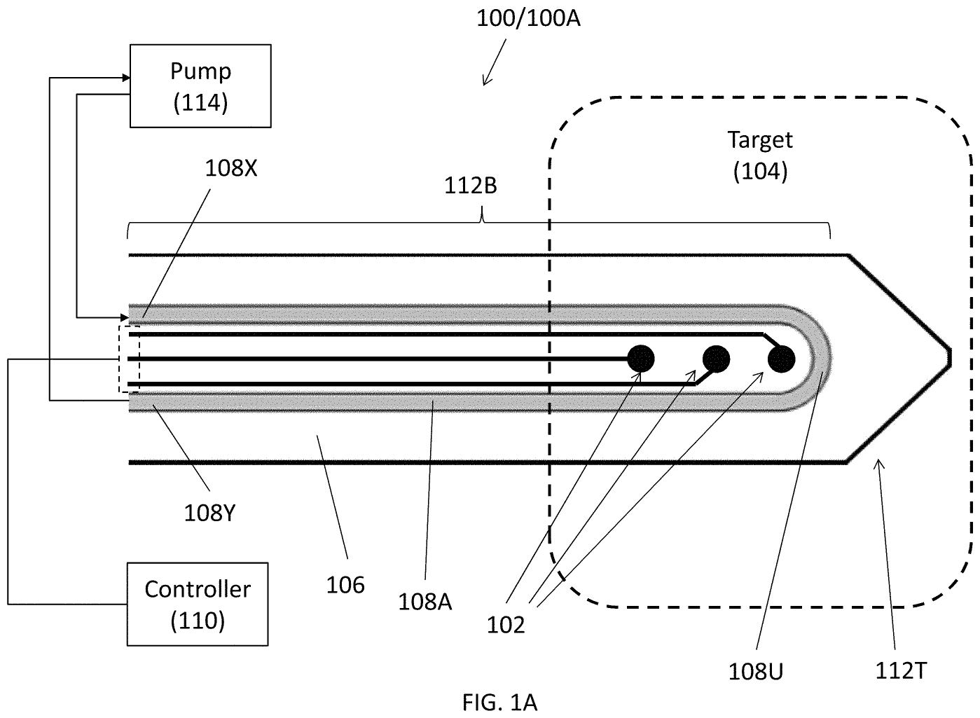

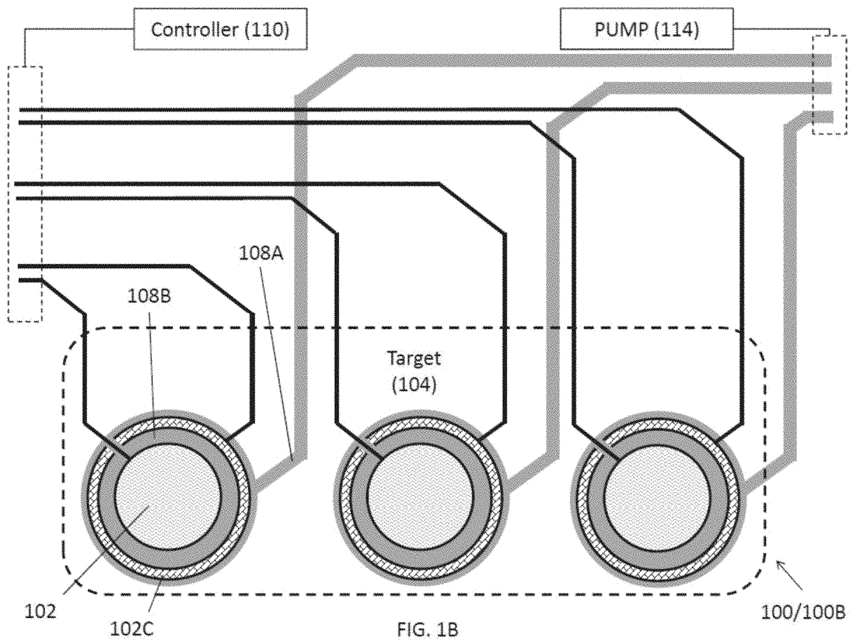

With reference to FIGS. 1A and 1B, there is shown an embodiment of an electromedical apparatus 100 comprising: an electrical component 102 arranged to facilitate an electrical signal transmission between an electrical device and a target 104; and a mechanical component 106 physically connected to the electrical component 102; wherein the mechanical component 106 includes a fluidic structure 108 arranged to modify at least one physical property of the mechanical component 106, so as to facilitate a deployment of the electromedical apparatus 100 on the target 104.

In this embodiment, the electromedical apparatus 100 comprises a plurality of electrodes or electrical contact pads which may transmit electrical signals between the target 104, such as a neuron cell or a receptor in a biological target, and an external controller 110. For example, control signal may be provided from the external controller 110 to stimulate the receptor cell in contact with corresponding one of the three contact pads of the electrodes 102, or the controller 110 may pick up or detect a neural signal generated by a neuron cell in contact with the electrodes 102.

Alternatively or additionally, other type of electrical or functional components such as sensors or electronic devices may be provided on the neural probe to provide functions other than signal transmissions. For example, temperature or pressure sensing electrodes may be provided to provide additional functions of the electromedical apparatus 100.

Referring to FIG. 1A, the electromedical apparatus 100 is a neural probe includes the electrodes 102 for transmitting electrical signals. The neural probe 100A includes an elongated body 112B (or a probe shank) and a probe tip 112T at an end of the elongated body. The external devices such as a signal controller 110 may connect to the neural probe 110A at the other end of the elongated body 112B.

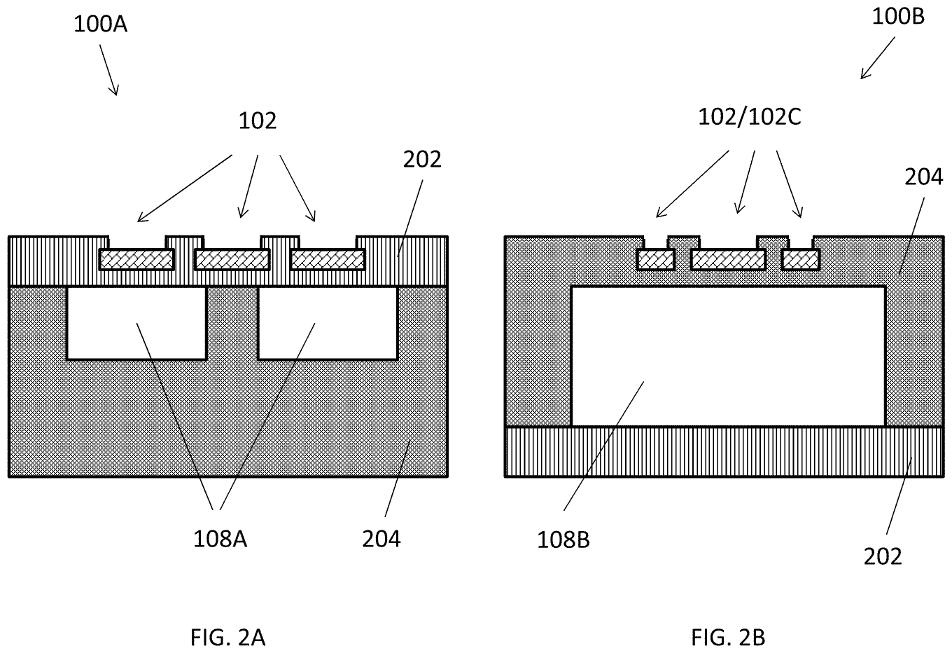

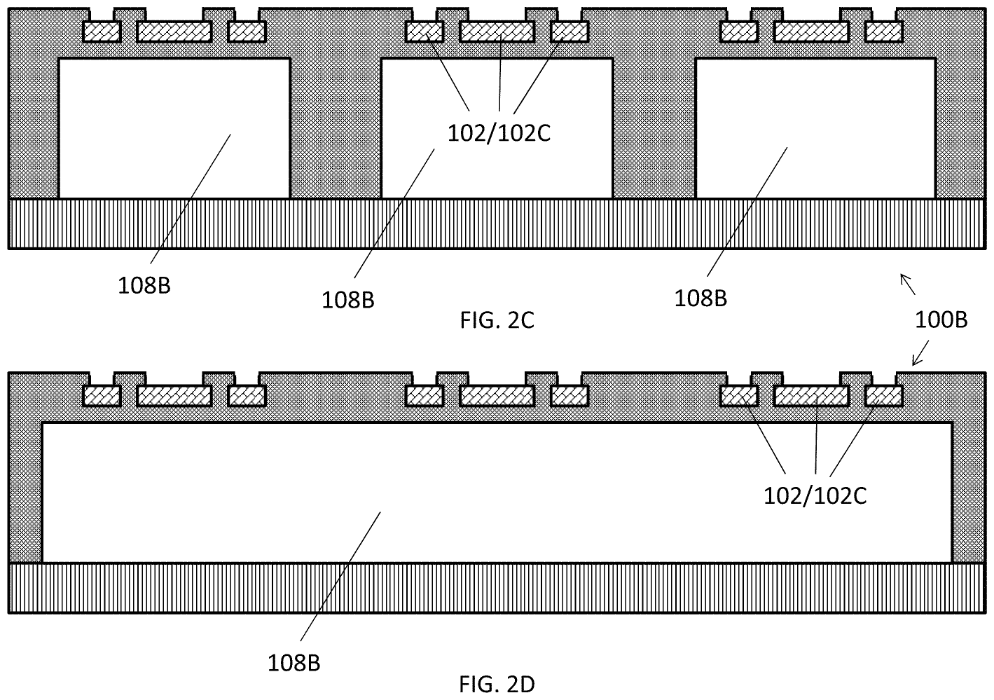

With reference also to FIG. 2A, the neural probe 100A comprises a mechanical component 106 which may be used for controlling a physical property, such as mechanical stiffness, of the neural probe 100A. As discussed earlier, by increasing the mechanical stiffness of the neural probe 100A, it may facilitate the deployment of the neural probe 100A into a biological target 104 at a precise position and to prevent the probe to buckle during the implantation of the neural probe 100A.

Preferably, the mechanical component 106 includes a fluidic structure 108, such as a fluidic channel 108A embedded in the probe body 112B of the neural probe 100A, for modifying the physical property of the probe. The position and dimension of the fluidic structure 108 are determined by the overall structure of the probe body 112B and according to the application of the probe. For example, the fluidic channel 108A may runs through a portion of the length of the elongated body 112B which needs to have a dynamically controllable stiffness.

In this embodiment, the mechanical component 106 comprises a multi-layered structure including a first layer of material 202 and a second layer of material 204, and the fluidic channel 108A is formed by encapsulating the trenches formed on the second layer of material 204 with the first layer of material 202. For example, a layer of polydimethylsiloxane (PDMS) may be patterned using an imprinting stamp to from the trenches and/or other three-dimensional structures, and then the PDMS layer 204 may be further bond to a layer of polyimide (PI) 202 using a PI/PDMS bonding method. Alternatively, the PDMS layer may be patterned using lithography.

As appreciated by a skilled person in the art, material other than PI or PDMS may be used to fabricate the electromedical apparatus 100, e.g. polymers or other mechanically flexible material may be used to form the probe body 112B. Preferably, the materials used are biocompatible for medical or biological applications. Besides, additional layers of material may be incorporated to form the multi-layer structure of the mechanical component 106 of the apparatus, which may further optimize the mechanical properties of the probe 100A, or to provide additional structural/functional components on the probe 100A based on different designs.

Referring back to FIG. 1A, the fluidic structure 108 further comprises an inlet 108X and an outlet 108Y, provided at one end of the probe body 112B, both in connection with the fluidic channel 108A, such that fluid may be injected to the fluidic channel 108A and is at least temporary held in the channel 108A. In addition, the fluidic channel further comprises a U-shaped portion 108U at or proximate to the second end, or adjacent to the probe tip 112T, of the elongated body 112B. In this configuration, injected fluids may run through the returning U-shaped path 108U and are discharged at the outlet 108Y which is provided at the same end of the inlet 108X. Alternatively, the returning path may include other shape or design, for example it may be preferable in some embodiments that the electrical contacts/wires do not overlap with an underneath fluidic channel.

Preferably, a pump 114 may be used to manipulate a flow of fluid through the fluidic channel 108A under a predetermine pressure. By continuously pumping or injecting a fluid into the fluidic channel 108A and at the same time varying an internal pressure of the fluidic channel 108A, the mechanical stiffness of the mechanical component 106 or the probe body 112B may be dynamically controlled. Alternatively, the fluidic channel 108A may be defined with a closed end in which fluids may be injected and extracted through the same inlet/outlet opening.

For example, the electromedical apparatus 100, such as a neural probe 100A referring to FIGS. 1A and 2A, may operate in at least two conditions. In a first condition, the mechanical stiffness of the mechanical component 106 is higher than that of the mechanical component 106 when the neural probe 100A operates in a second condition, in which the mechanical component 106 may prevent bending or buckling of the neural probe body 112B during the deployment or implantation of the neural probe 100A on the target 104.

After disposing the neural probe 100A in the desired position, the internal pressure in the channel 108A may be decreased, such as by discharging the fluidic from the fluidic channel 108A, thereby “returning” the probe 100A to its flexible state. The internal pressure may be again increased or controlled by pumping in a fluid in the fluidic channel 108A, to facilitate moving the implanted neural probe 100A to a second position, without repeating the entire deployment process.

The soft neural probe body may reduce glial cell formation in the inserted region and prevent signal quality degrading. In some occasions, glial cells may form during stimulation or neural activity recording, the neural probe position may be adjusted by applying fluidic pressure inside the fluidic channel 108A, thereby facilitating relocation of the probe 100A to an area without damaged cells.

Referring to FIGS. 1B, 2B and 2C, in an alternative embodiment, the electromedical apparatus 100 is a micro-electrode array (MEA) apparatus 100B. For example, the MEA 100B comprises an electrical component 102 having three electrodes arranged in a row, and a fluidic structure 108 comprises an array of fluidic cavities 108B, with each of the fluidic cavities 108B connected to a fluidic channel 108A for transferring fluidic into and out of the fluidic cavities 108B, e.g. with the help of an external pump 114 in communication with the fluidic channel 108A.

The mechanical component 106 may be used improve a physical contact between the electrical component 102 and the target 104, for example when the MEA 100B is deployed to contact with a biological target 104 with non-planar surface such as on a retinal surface of an eye. The retinal surface may include a complex topography which is not perfectly fit with the curvature of a substrate of the MEA 108B, which may results in gaps between the electrodes 102 and the retina cells, thereby causing bad contacts between the electrodes 102 and the retina at or around those regions with gaps between the two.

In this example, the physical property of the fluidic structure 108 includes a physical dimension, i.e. internal volume of each of the fluidic cavities 108B. By injecting fluids into the internal cavities 108B through the associate fluidic channels 108A, the internal pressure and thus the internal volume of the cavities 108B may be dynamically controlled. Preferably, the physical dimension is modified in response to a variation of an internal pressure in the fluidic channel 108A and/or the fluidic cavity 108B.

Referring to the cross sectional view of a portion of the MEA apparatus 100B as shown FIGS. 2B and 2C, the working principle of such control is similar to that of filling a balloon to increase the volume of the balloon, therefore eliminating or at least decreasing the size of the gap between the contact electrode 102 and the target 104/retinal surface, and hence improving the physical contact with the target 104 by pushing the electrodes 102 towards the target 104.

For example, after disposing the micro-electrode array apparatus 100B on the target 104, the operator may control the internal pressure of each of the plurality of fluidic cavities 108B so as to improve the physical contact between the electrical component 102 and the target 104 at each respective portion of the electrical component 102.

Preferably, each of the electrodes 102 in the electrical component 102 aligns with a corresponding fluidic cavity 108B in the mechanical component 106, so as to facilitate a pixel-to-pixel manipulation of the individual cavities. Alternatively, the two arrays may be offset or misaligned to each other, or the size/dimension of the two arrays may be different.

In response to the variation of the internal pressure in the fluidic cavity, the fluidic cavities 108B may move a respective portion of the electrical component 102 with respect to the target during the deployment of the electromedical apparatus 100 on the target. Therefore, the two arrays in the electrical and the fluidic cavities 108B are not necessarily the same. For example, referring to FIG. 2D, a fluidic cavity 108B with substantially bigger planar area may be responsible for improving a larger portion of the MEA 100B covering multiple electrodes 102 may be defined, and therefore may require fewer individual fluidic channels 108A in the apparatus. In addition, the shape of the fluidic cavities 108B may not be the same and may include shapes other than round shape in different designs.

Optionally, the electrical component 102 further comprises a counter electrode 102C adjacent to each electrode in the array of electrodes 102. The counter electrode 102C may be used for estimating a position of the electrode with respect to the target 104 so as to evaluate the physical contact between the electrical component 102 and the target 104. For example, after disposing the micro-electrode array apparatus 100B on the target 104, by measuring an electrical resistance between the ring-shaped counter electrode 102C and the round electrode 102, a high resistance may represent a bad contact (due to a large gap) between the electrodes 102 to the target surface, then the corresponding fluidic cavities 108B may be adjusted to push the electrodes 102 towards the target surface to improve the physical and hence the electrical contacts, and the quality of such contacts may be simultaneously monitored using the counter electrodes 102C.

In addition, the ring-shaped counter electrodes 102C may create an electrical isolating boundary during retinal stimulation to prevent cross talk of adjacent stimulating/main electrodes 102 due to electrical current leakage, for example by grounding all the counter electrodes 102C during operation of the MEA 100B.

Alternatively, the counter electrodes 102C may be defined with different shape and dimension, provided that the counter electrode is disposed adjacent to the main electrode for signal transmission.

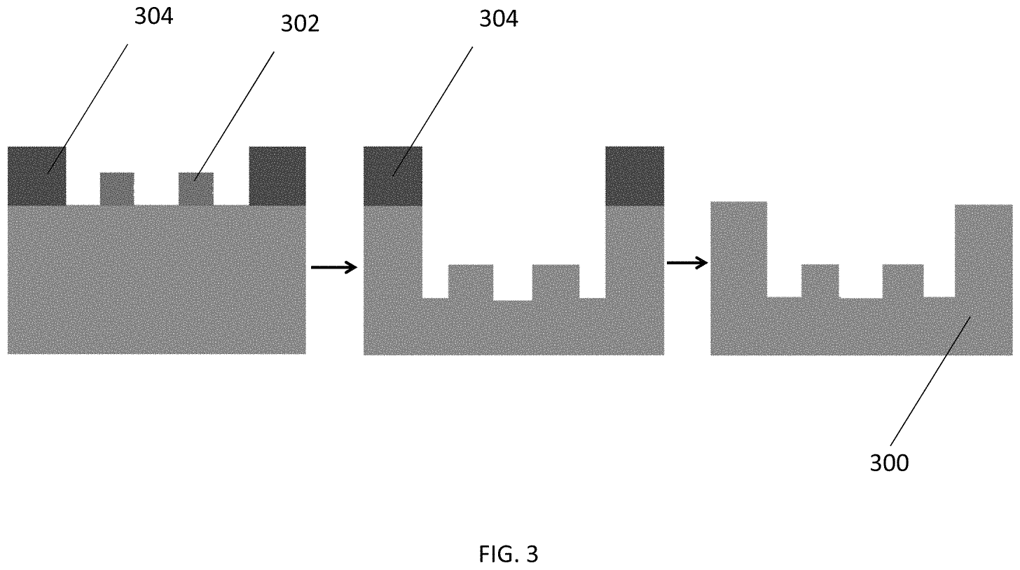

With reference to FIG. 3, there is shown an example embodiment of a fabrication of an imprinting stamp 300 for use in the subsequent fabrication of the fluidic structures 108 in the electromedical apparatus 100 such as the neural probes 100A and the MEA apparatus 100B as discussed above.

In this example, the imprinting stamp 300 may be used for patterning PDMS using a reversal imprinting method, in which PDMS material may be deposited on the stamp 300, then the cured PDMS material may be detached and further transferred to another substrate or layer of material.

For example, silicon (Si) stamp 300 with 3D patterns of trenches or microchannels may be fabricated for reversal imprinting PDMS channel plate 204, the PDMS layer may be stacked and chemically bonded on the PI cover plate 202 using 1 μm thick poly(methyl methacrylate) (PMMA) as an intermediate bonding layer. With such PMMA layer, chemical bondings form between he PI and PDMS layer and firmly combine the two layers of material. Alternatively, the first layer of material and the second layer of material are combined via a layer of adhesive material.

For the channel shape in Si stamp, 1 μm thick AZ nLOF 2070 (diluted) negative photoresist 302 may be spin-coated on Si at 3000 rpm for 60 s and soft baked at 130° C. for 60 s on a hot plate. The photoresist 302 may be patterned by UV exposure and post-baked at 130° C. for 60 s. After the negative photoresist is developed, a 5 μm thick AZ 6130 positive photoresist 304 may be spin-coated at 3000 rpm for 60 s and baked at 90° C. for 10 min. After patterning the positive photoresist 304, the Si stamp 300 is ready to be dry etched.

A deep reactive ion etching (DRIE) (LPX ICP LE0729, SPTS) system may be used to etch the Si stamp in 2 steps. Initially, Si material of a thickness of 20 μm is etched to define the channel depth and the remaining negative photoresist 304 may be removed using O₂ plasma. The Si stamp may be further etched for an additional 40 μm to realize 60 μm total channel plate thickness. The remaining positive photoresist 302 may then be removed in acetone for 10 min.

With the Si stamp or mould 300, the fabrication of the electromedical apparatus 100 may follow with the steps of defining a first layer of material 204 with a fluidic structure 108; combining the first layer of material 202 with a second layer of material 202 to form a multi-layer structure defining a mechanical component 106 with the fluidic structure 108; and providing an electrical component 102 to physically connect with the mechanical component 106.

Using a reversal imprint process, the Si stamp 300 may be coated with trichloro(1H, 1H, 2H, 2H-perfluorooctyl) silane (FOTS) (Sigma-Aldrich, 97%), which acted as an anti-sticking layer 305. A 60 μm thick 10:1 (weight ratio, w/w prepolymer to curing agent) PDMS 204 (Sylgard 184, Dow Corning) may be spin-coated at 1000 rpm for 60 s on the Si stamp 300 and reversely imprinted onto a handling substrate 306, such as another Si substrate at 130° C. and pressure of 40 bar for 10 min.

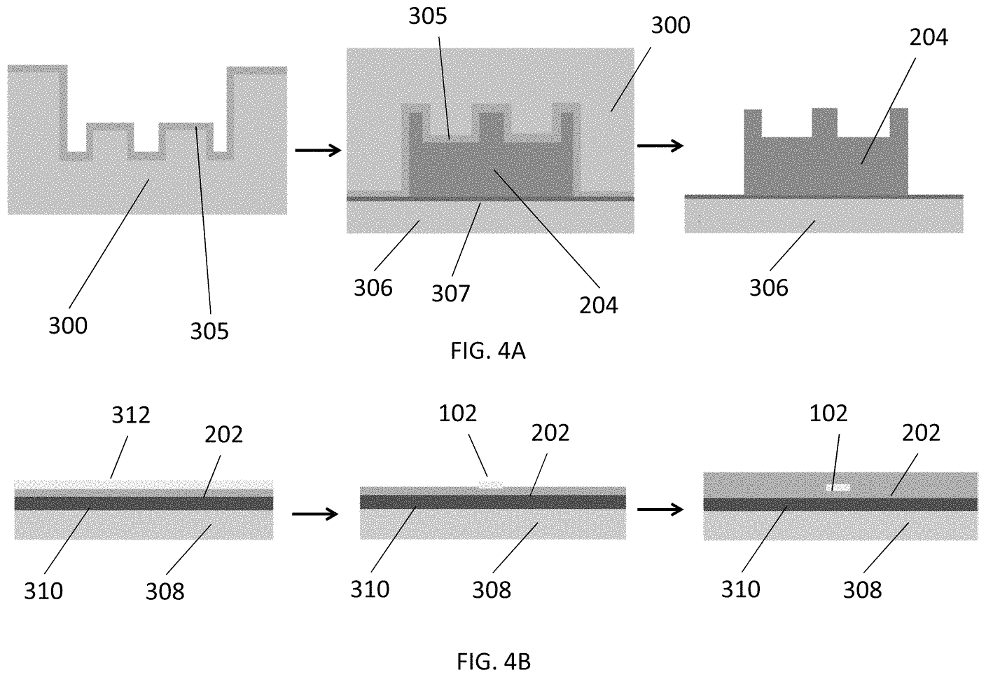

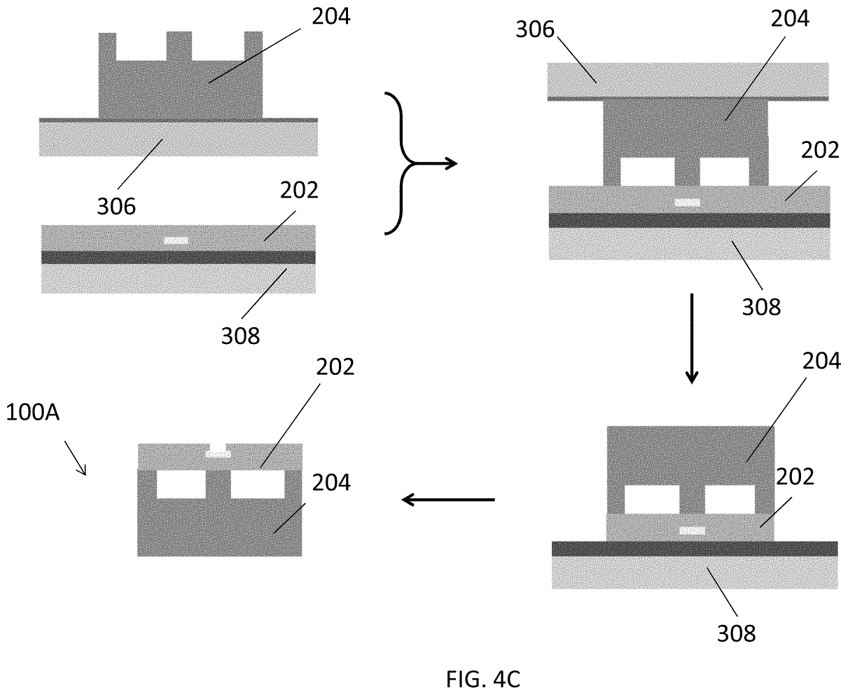

With reference to FIG. 4A, on this handling substrate 306, the surface may be coated with 1:4 FOTS/methacryl oxypropylene trichlorosilane (MOPTS) 307 to provide higher surface energy than the FOTS coated Si stamp 300, such that the PDMS layer coated on the stamp may be transferred to the handling substrate 306 when the stamp is released from the substrate after pressing the PDMS layer onto the substrate.

The ratio of FOTS and MOPTS may be tuned according to the desired surface energy on both the stamp and the handling substrate. Alternatively, other coating material may be used to modify the surface energy of the surfaces of the stamp or the substrates as desired.

Due to different surface energy between the Si stamp 300 and the Si handling substrate 306, the PDMS-based channel plate 204 could be detached from the Si stamp 300, and transferred to the Si handling substrate 306, thereby providing a PDMS layer 204 with the fluidic structures 108 formed on it.

The fluidic channels 108A are defined by encapsulating the trenches formed on the PDMS layer 204 and encapsulated with an additional layer of material, such as a layer of polyimide (PI) 202. For example, referring to FIG. 4B, the fabrication of a PI covering layer 202 may start with a clean glass or silicon substrate 308. 1 μm PMMA 310 may be first spin-coated on the glass substrate at 3000 rpm and baked at 240° C. for 30 min to facilitate the final release of the neural probe 100A from the glass substrate 308. Then 15 μm PI 202 may be spin-coated at 1000 rpm for 60 s and baked at 180° C. for 5 h, followed by 240° C. for 4 h.

The electrical component 102 or the electrodes may be provided on the first or the second layer of material, based on different application of the electromedical apparatus 100. For example, to fabricate a neural probe 100A, the metal layer 312, which may include multiple layers of Cr and Au, may be deposited on PI 202, and the metal layer 312 may be patterned using lithography and etching method, and then the patterned metal layer 312 is subsequently covered by an additional layer of PI 202. An additional 1 μm thick PMMA 310 may be spin-coated at 3000 rpm for 60 s as an intermediate bonding layer with the PDMS channel plate 204.

Alternatively, other suitable metal, such as Pt may be used to define the electrodes or other electrical components of the electromedical apparatus 100.

With reference to FIG. 4C, PDMS channel plate 204 or the second layer of material may be treated by an O₂ plasma (Plasma-Term 790 Series) using 25 sccm O₂, 65 W stage power, and 700 mTorr chamber pressure for 20 s. The PDMS channel plate 204 and the PI cover plate 202 may then be bonded together with low pressure for 2 min, followed by baking at 130° C. for 1 h. An inductively coupled plasma (ICP) system (Oxford Instruments, Plasmalab 80 Plus) may be used to dry etch 1/15 μm PMMA/PI at 10 sccm O₂, 60 W stage power, 450 W Source power, and 10 mTorr chamber pressure for 15 min. PMMA and PI etch rate may be set at 1.0 and 0.8 μm/min, respectively, with the PDMS layer 204 as an etch mask. Finally, the entire PDMS/PI neural probe 100A may be detached from the glass substrate 308, for example by stripping away the PMMA layer 310 between the glass substrate 308 and the PI layer 202, and lastly exposing the electrical contacts of the electrical component 102 by etching some PI covering the electrodes in the PI layer 202.

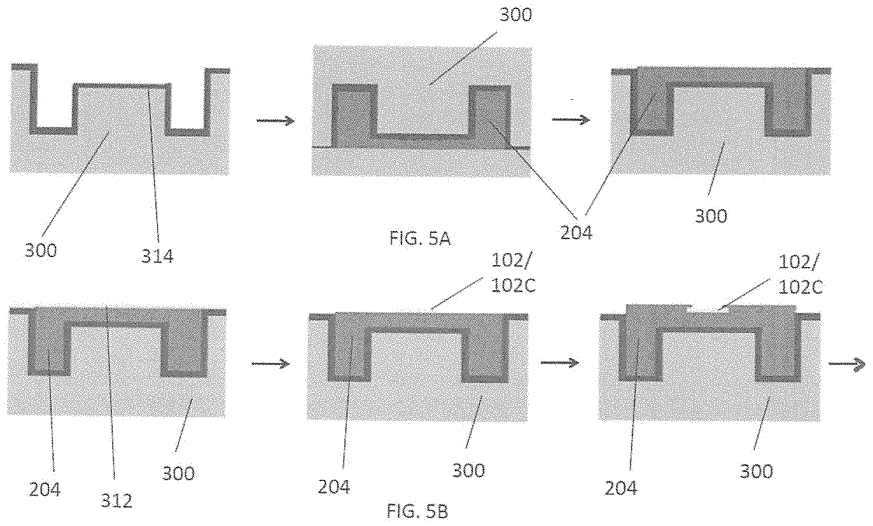

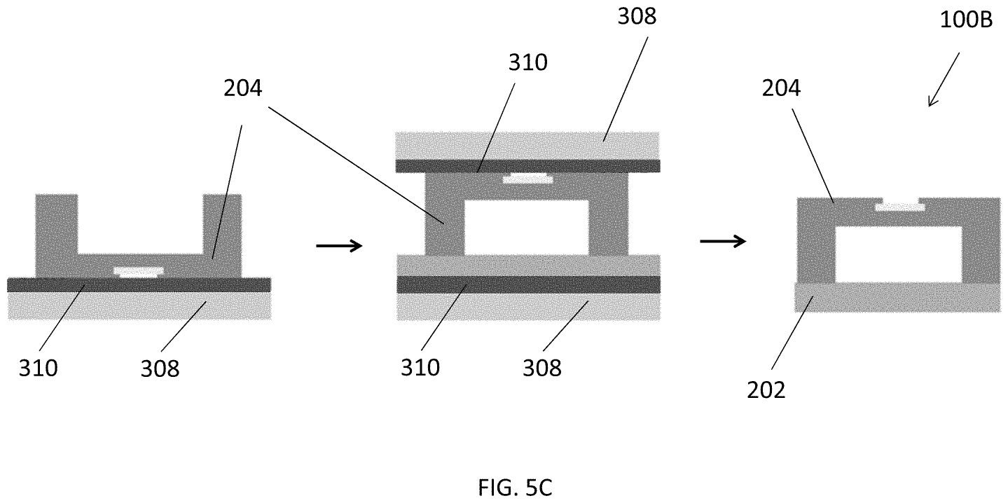

With reference to FIG. 5A to FIG. 5C, there is an alternative embodiment of the method for fabricating the electrical apparatus, such as an MEA apparatus 100B, including the fluidic structure 108 and the metal electrodes 102. A main different between the process for fabricating the MEA 100B and the neural probe 100A is that the electrodes 102 are provided on the PDMS layer 204 (second layer of material) in the MEA 100B instead of on the PI layer 202 (first layer of material) in the neural probe 100A. On the other hand, the reversal imprinting process, i.e. forming the fluidic structure 108 on the PDMS layer 204, stacking and bonding of the two layers to complete the mechanical component 106 of the apparatus 100 are similar in two processes.

In this example, the process also starts with the preparation of the stamp 300, preferable made of Si or SU-defining the structures of the microfluidic channels 108A and the fluidic cavities 108B. Firstly, referring to FIG. 5A, silane 314 may be coated on the Si stamp 300, and PDMS may be applied on the Si mould 300 to form the PDMS layer 204 with the fluidic cavities 108B and the fluidic channels 108A.

Secondly, the PDMS layer 204 on the Si stamp 300 (detached from a Si substrate with FOTS/MOPTS coated thereon) may be further deposited with a layer of metal 312 such as Cr, Au and/or Pt, followed by patterning the metal layer 312 by photolighography and etching of the metal layer 312 to define the metal electrodes 102/102C on the PDMS surface. The patterned electrodes 102 may be further protected by a PDMS passivation layer followed by an electrode opening step using dry etching. Alternatively, the electrode opening step may be performed in the last step after combining the PDMS layer 204 with the PI layer 202 and releasing the combined layers from the glass handling substrates 308.

Thirdly, referring to FIG. 5C, the PDMS layer 204 with the 3D structure and the electrodes 102 may be transferred to a handling glass substrate 308 with PMMA coated thereon after detaching and releasing the PDMS layer 204 form the Si stamp 300. Then the PDMS layer 204 on the handling glass substrate 308 may be flipped again on a PMMA/PI/PMMA/glass substrate followed by a PI/PDMS bonding process to stack and combine the PDMS 3D structure on the PI layer 202.

Lastly, the MEA apparatus 100B including the PDMS layer 204 and the PI layer 202 may be released from the two glass substrates 308 by stripping the PMMA layers coated on glass, and the PI layer 202 may be further etched using the PDMS layer 204 as the etching mask.

Apart from PDMS and PI, other materials may be used to fabricate the mechanical components in the electromedical apparatus. Preferably, the material is biocompatible, which may include most of the polymers. Alternatively, non-polymeric materials such as Si or quartz may be used if coated with biocompatible materials. Additionally or optionally, the first and second layers of material may include materials which may have other chemical or mechanical properties, such as being mechanically flexible and/or electrically insulating.

Preferably, one side (layer) of the probe is more flexible and the other side is more rigid. For the more flexible layer, for example, polyethene other than PDMS may be used. Polymers that contain long molecules that lie side by side can uncoil and slide past each other, making the material flexible.

For the more rigid layer of material, parylene and SU-8 other then PI could be used. Cross-linking is where the polymer chains are chemically joined together in places, by covalent bonds. The polymer molecules cannot slide over each other so easily. This makes materials tougher and less flexible, and they may not be easily stretched.

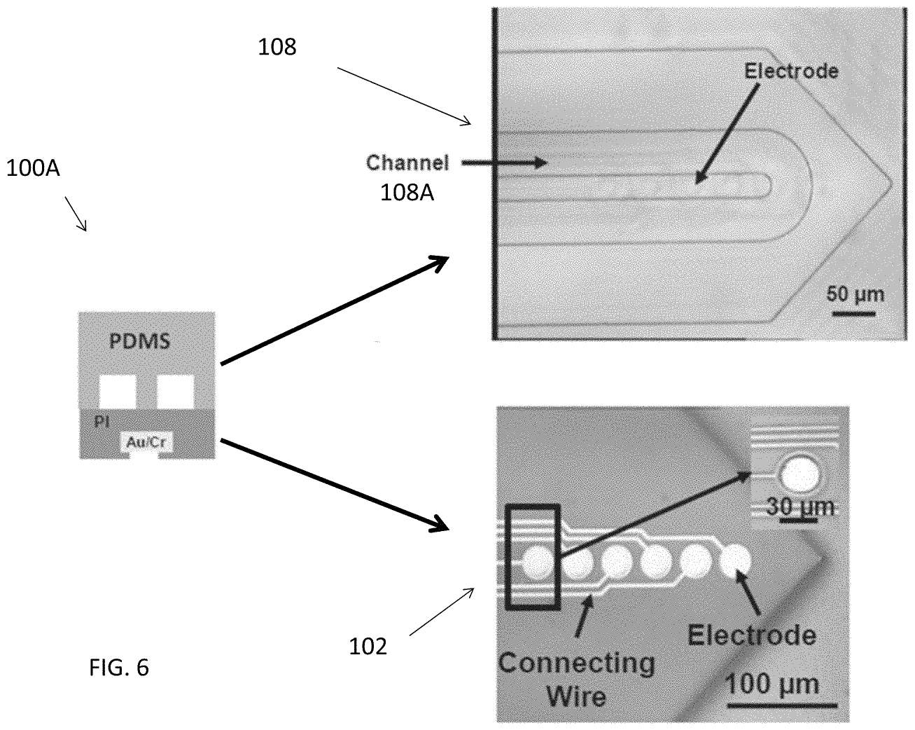

With reference to FIG. 6, there is shown a cross-sectional view of the neural probe 100A and microscopic photographic images showing the mechanical and the electrical components 102 near the probe tip section. In this example, each of the electrical contact pads is round shape with a diameter of 30 μm, and the channel width of the fluidic channel 108A is about 50 μm.

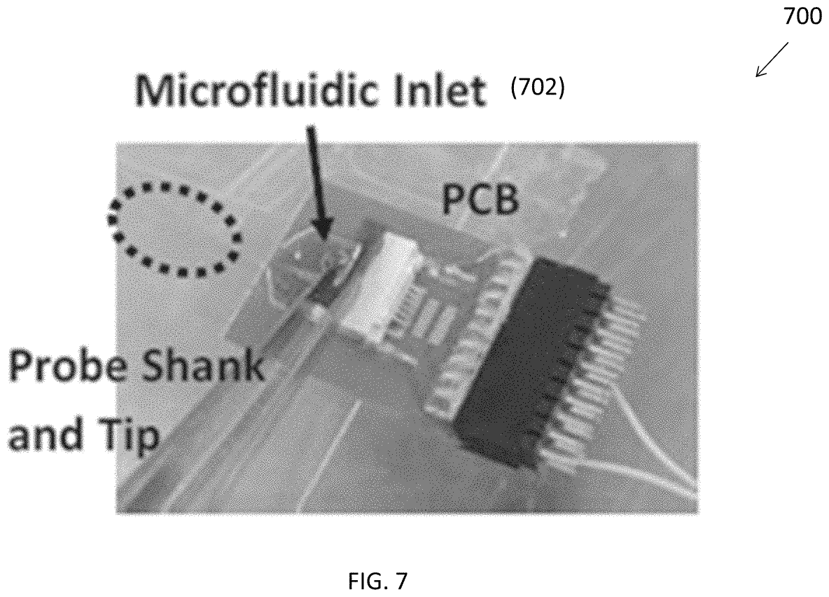

With reference to FIG. 7, there is shown a controller board 700 for use in connecting the electrodes 102 on the neural probe 100A to an external controller 110, a microfluidic inlet 702 is provided on the controller board for connecting the inlet 108X and to the pump 114 for injecting the fluid into the fluidic channel 108A for dynamically adjusting the internal pressure of the fluidic channel 108A.

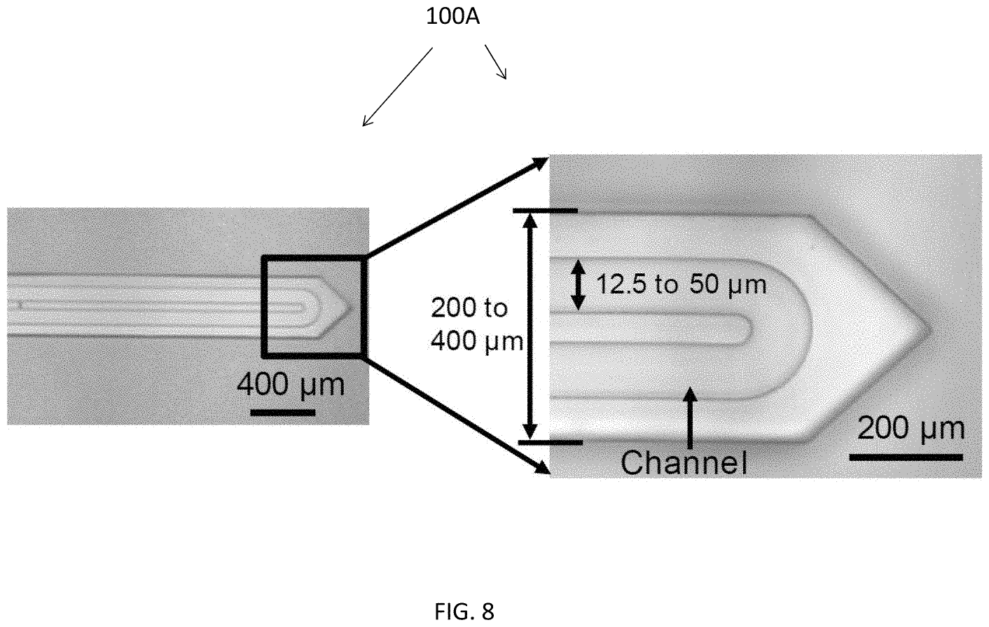

With reference to FIG. 8, there is shown a photographic image of an alternative example neural probe 100A (without the electrodes) fabricated for further evaluation of the performance of the mechanical components 106 of the neural probe 100A.

In the experiment, the embedded microchannels formed a ‘U’ shape in the neural probe near the probe tip, with single channel width of 12.5, 25, and 50 μm on each side of the U-shaped returning path, the total (in and out) channel width was 25, 50, and 100 μm, respectively. In addition, the width of the neural probes varied from 200 to 400 μm. It is devised that the channel width and fluidic pressure in the channels determined the flexibility of the neural probes, which could be optimized for implantation and reduced tissue damage.

The inventors has studied the mechanical performances of the fabricated neural probe, by measuring the flexibility of these neural probes using the buckling test that resembles the probe insertion into the targeted brain tissues.

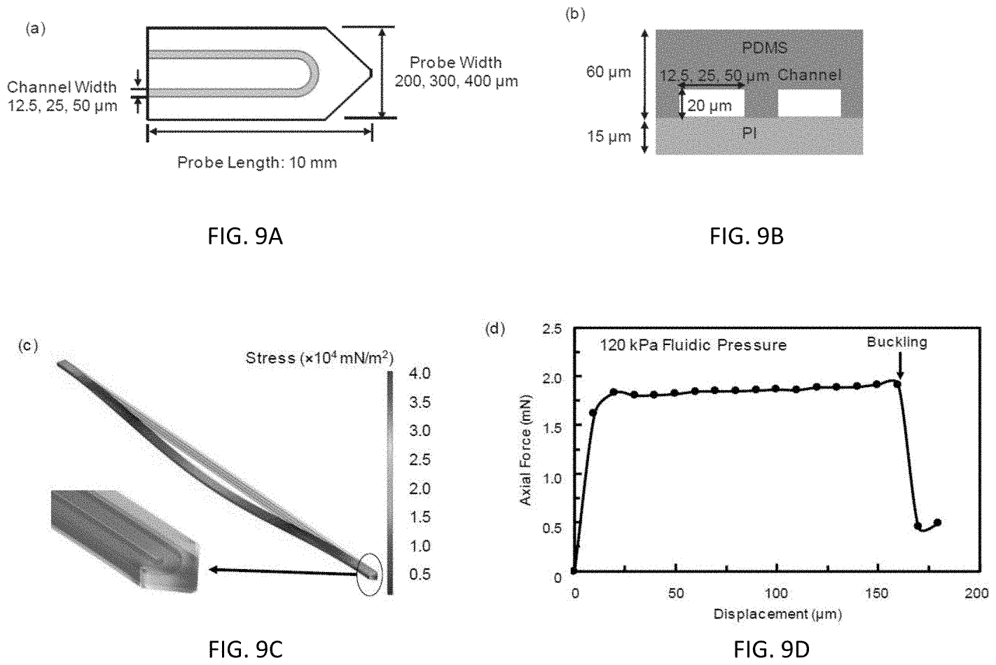

In the experiment, neural probes with different materials and dimensions were simulated using the FEM-based COMSOL MULTIPHYSICS software. With reference to FIGS. 9A and 9B, different neural probes were modeled in 3-dimensional Cartesian coordinate system with 2-dimensional views. The probe head was assumed to be fixed during the buckling. However, the probe tip was assumed to be sharp and could only move along the probe shank direction and could not slide along the surface.

With reference to FIG. 9C, there is shown the the simulated von Mises stress distribution and the deflection of the probe along the probe shank. The critical buckling force was determined by measuring the force exerted by the neural probes on a microbalance (Sartorius BP 211D, Sartorius AG, Germany) with resolution of 0.01 mg. At the initial contact between the probe and the balance, the force value was set to zero.

With reference to FIG. 9D, there is shown the changes of the axial force during the buckling test for a 60/15 μm thick PDMS/PI neural probe with probe width of 400 μm, channel width of 12.5 μm, and 120 kPa fluidic pressure in the microchannel. During the buckling test, after the probe tip contacted the microbalance surface, the probe shank gradually deformed as the probe was pushed down onto the microbalance by the induced force. At the threshold force of 1.9 mN, the probe buckled and the probe tip moved. All the critical buckling force was obtained based on the first buckling mode.

The following table shows a comparison of a neural probe in accordance with embodiments of the present invention with other example neural probes in controlling neural probes flexibility. It is shown that the neural probe of the present invention is only one that provided dynamic control of probe flexibility using adjustable fluidic pressure in the microchannels. In addition, the neural probes of the present invention may be designed to include different materials, such as PDMS and PI polymers, and with various probe dimensions. Advantageously, the neural probes may be optimized to the desirable low flexibility needed for implantation and high flexibility needed for in vivo operation with low tissue damage.

| | | | Channel | Flexibility | Buckling | | Insertion |
|---|---|---|---|---|---|---|---|
| | Materials | Probe Size | Size | Control | Force | | Force |
| The | PDMS/PI | 60 μm thick; | 12.5-50 | PDMS/PI; | 0.5-2.1 | mN | 0.8 mN |
| present | | 200-400 μm | μm wide; | fluidic | | | at 60 kPa |
| invention | | wide; 1 cm | 20 μm | pressure in | | | |
| | | long | deep | channels | | | |
| Example | Parylene-C | 24 μm thick; | — | Biodegradable | 2.6-300 | mN | 8-23 mN |
| A | | 120 μm wide; | | PEG and silk | | | |
| | | 3 mm long | | | | | |
| Example | Si/Parylene | 100 μm | — | Parylene tube | 540 | mN | — |
| B | tube | thick; 120 | | dimension and | | | |
| | | μm wide; 3 | | location | | | |
| | | mm long | | | | | |
| Example | Si | 15 μm thick; | — | Breakable Si; | 1.7 | mN | — |
| C | | 100 μm wide; | | biodegradable | | | |
| | | 5.5 mm long | | polymer | | | |
| Example | Parylene | 20 μm thick; | — | Parylene | 0.5-1.3 | mN | — |
| D | | 200 μm wide; | | stiffener | | | |
| | | 2 mm long | | | | | |
| Example | Parylene | 20 μm thick | 50-200 | Biodegradable | 12 | mN | — |
| E | | | μm wide; | PEG in | | | |
| | | | 10 μm | channel | | | |
| | | | deep | | | | |

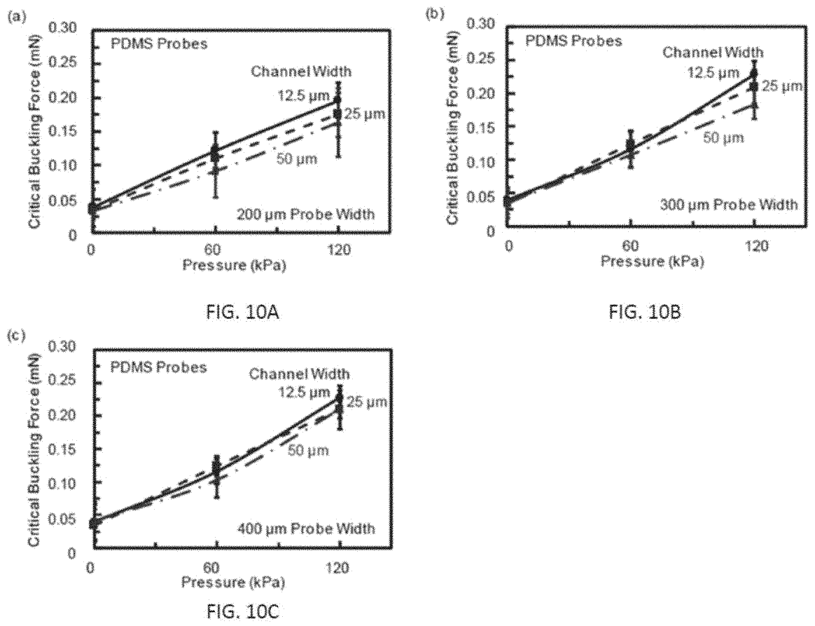

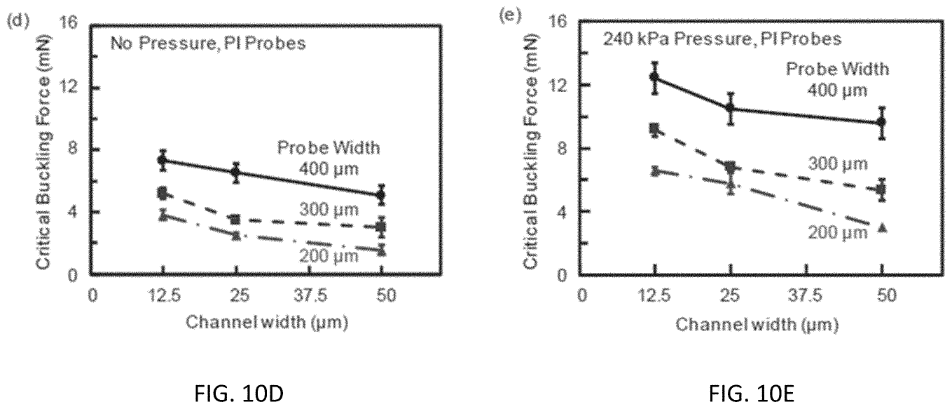

With reference to FIGS. 10A to 10C, there is shown the experimental results of critical buckling force for the PDMS-based neural probes with 200, 300, and 400 μm probe width, respectively, and FIGS. 10D and 10E illustrate the results for PI-based neural probes. In can be concluded that the critical buckling force for these PDMS-based probes were much smaller than the PI-based neural.

For 400 μm wide probes with 12.5 μm channel width, the critical buckling forces were found to be 0.06 and 7.30 mN for PDMS- and PI-based neural probes, respectively, with no fluidic pressure inside the channels. As channel width increased, the critical buckling force decreased for both types of probes. However, this effect was more significant for PI-based probes. The measurements also showed that the critical buckling force in the PDMS-based probes did not change significantly for probe widths varying from 200 to 400 μm due to PDMS's hyperelastic properties. Further increase of the fluidic pressure to 240 kPa inside the embedded channel would cause channel deformation and swelling since PDMS is relatively flexible when compared to PI.

However, the higher flexibility of the PDMS material that allows the deformation of the fluidic structure and hence the dimension of the body of the apparatus may be desired in the MEA apparatus. Details of the MEA apparatus will be discussed later in the disclosure.

Referring to FIGS. 10D and 10E, the critical buckling forces for 60 μm thick PI-based neural probes with different probe width and channel width are shown in without and with 240 kPa fluidic pressure. The critical buckling force for these PI-based neural probes decreased with decreasing probe width or increasing channel width. Fluidic pressure inside the microchannel could increase the critical buckling force significantly.

For example, for 400 μm wide PI probe and 12.5 μm wide channel, the critical buckling force was 7.30 mN without fluidic pressure and it increased to 12.40 mN when 240 kPa fluidic pressure was applied. Similar increases were observed for 200 and 300 μm wide probes that the critical buckling force could be increased by applying fluidic pressure in the channels.

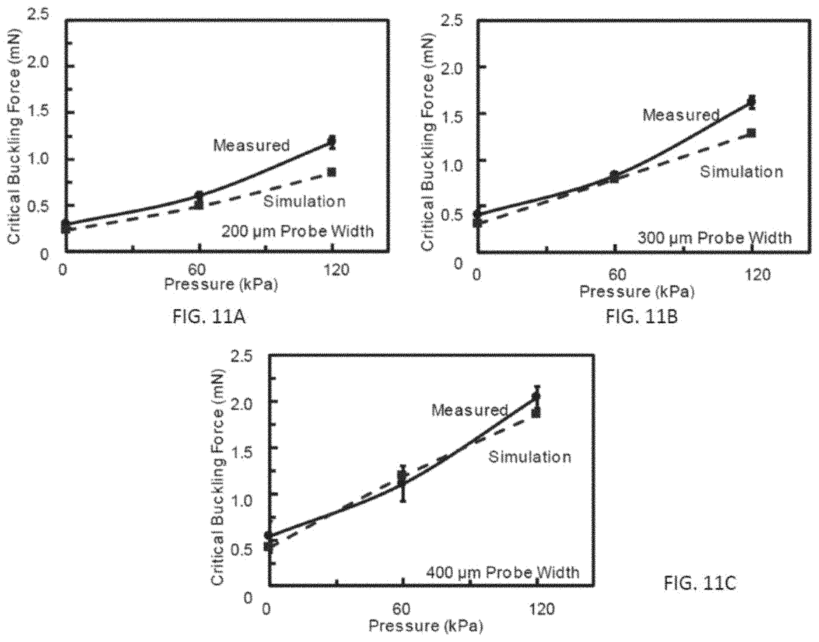

With reference to FIGS. 11A to 11C, there is shown the experimental and simulation results of the critical buckling force for the 60/15 μm PDMS/PI neural probe with 12.5 μm channel width. For 200 μm wide neural probe with 12.5 μm wide channel, the critical buckling force was 0.29 mN with no fluidic pressure and it increased to 0.60 mN when the pressure was increased to 60 kPa. As the pressure was increased further to 120 kPa, the critical buckling force continued to increase to 1.17 mN. The measured critical buckling force for neural probes at different pressures closely matched with the simulated results. Similarly, referring to FIGS. 11B and 11C, the measured and simulated results for 300 and 400 μm wide probes followed the same trends.

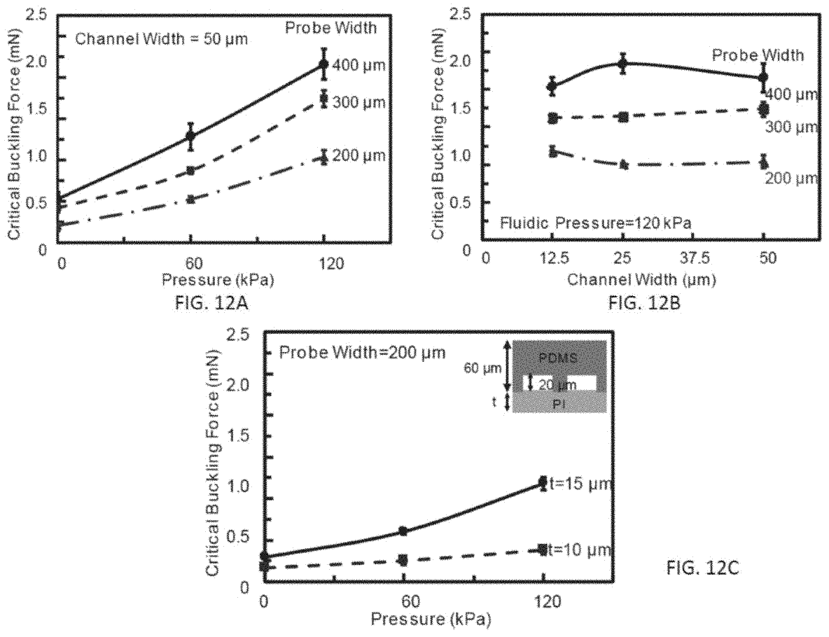

With reference to FIGS. 12A and 12B, there is shown the effects of fluidic pressure on critical buckling force for neural probes with different channel widths and probe widths. The probes became stiffer with wider probes width, but the channel width had little effects on the critical buckling force. Channel width did not change the critical buckling force significantly because the probes consisted of 60 μm thick PDMS and only 10 or 15 μm thick PI layer with the channels. However, referring to FIG. 12C, by changing the PI layer thickness from 10 to 15 μm the neural probes become stiffer.

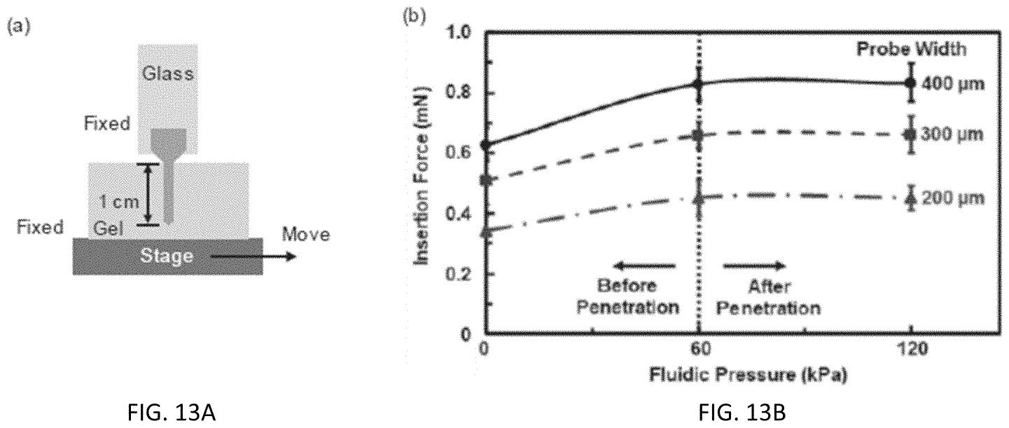

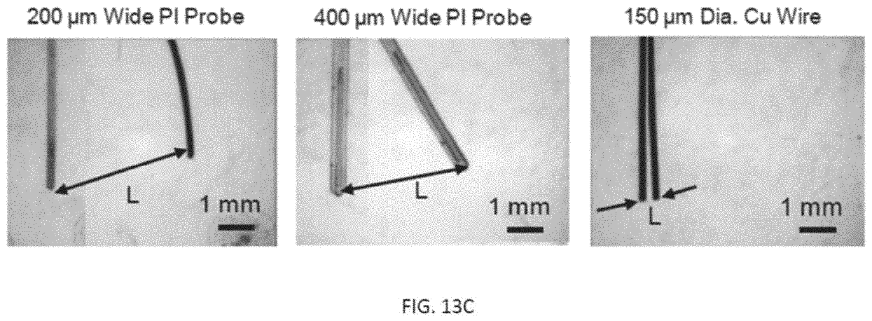

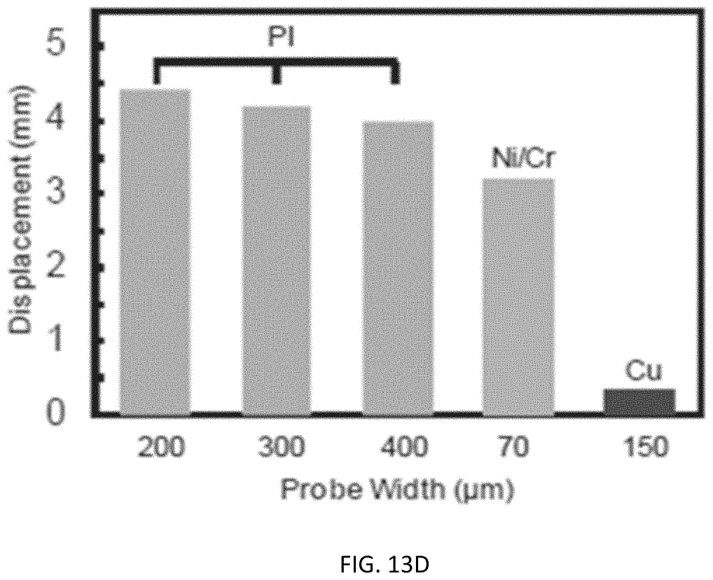

With reference to FIG. 13A, in another experiment, the fabricated probes were inserted into a 0.6% agarose gel that mimicked the brain tissue mechanical properties. With reference to FIG. 13B, the plot illustrates measured insertion force as a function of fluidic pressure in the channels. The neural probes could be inserted into the gel when the applied force was large enough to shear through the agarose gel.

When no fluidic pressure was applied in the embedded microchannels, the probes were not able to penetrate into the gel. The insertion force was found to be 0.34, 0.51, and 0.63 mN for 200, 300, and 400 μm probe width, respectively, with no pressure inside the channels. The applied force deformed the surface of the gel without shearing through, therefore the probes were not able to penetrate into the gel.

Referring to FIG. 13, by increasing the fluidic pressure to 60 kPa, the probes may shear through the gel surface with insertion force of 0.45, 0.66, and 0.83 mN for 200, 300, and 400 μm probe width, respectively. Further increasing fluidic pressure to 120 kPa caused the probes to penetrate into the agarose gel. At higher pressure beyond 120 kPa, PDMS started to swell and became undesirable.

In order to characterize the flexibility of the neural probes, PI probes with width of 200, 300, and 400 μm were fabricated. These probes were inserted into the gel and the gel was moved laterally, while the probe displacements were tracked using a camera. Nickel/chromium (Ni/Cr) and copper (Cu) metal wire probes with a diameter of 70 and 150 μm, respectively, were used to compare the probes flexibility. Since a minimum force of 0.5 mN was needed to successfully implant the neural probes into the brain tissue.

As discussed earlier, the PDMS probes had critical buckling force below 0.5 mN and they were too soft to be inserted into the agarose gel. Therefore, the probe displacement measurements were performed in PI and metal wire probes. With reference to FIG. 13C, the micrographs of the probe positions inside the gel at their starting and ending positions after the gel was moved laterally by 4 mm are shown, and the results are further illustrates in the plot as shown in FIG. 13D, which shows the probe displacement, L, for different PI and metal wire probes.

Referring to FIG. 13D, PI probes with width of 200, 300, and 400 μm had displacement that decreased with increasing probe width. In comparison, the displacement for the Ni/Cr and Cu wire probes were 3.21 and 0.35 mm, respectively. These results show that the polymer-based neural probes were more flexible than the metal wire probes, which have the advantages of inducing less tissue damage due to brain micro-motions.

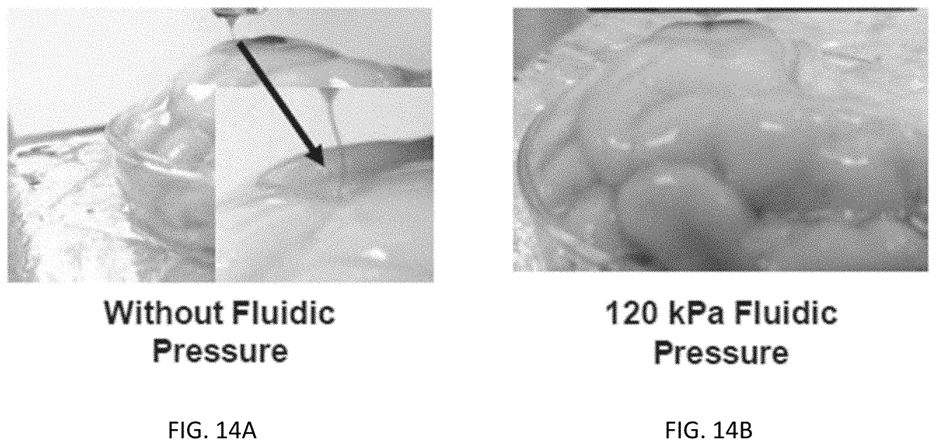

With reference to FIGS. 14A and 14B, there is shown another experimental result of deployment of neural probes in a rat brain without and with 120 kPa fluidic pressure respectively. It is obvious that the neural probe without a high fluidic pressure was not able to penetrate through the surface of the brain due to buckling. In contrast, the probe with 120 kPa fluidic pressure was successfully implanted.

Both the neural probe 100A and MEA 100B has embedded microfluidic channels 108A where fluidic pressure can be applied. On the flexible neural probe, electrode 102 and connecting wires were patterned on the polyimide (PI) 202, which is a non-stretchable material, only the overall probe rigidity is designed to be adjusted without deforming the metal layer. On the other hand, in MEA apparatus 100B, metal layer 312 was patterned on PDMS 204 above the microcavity 108B, referring to FIG. 5C. When fluidic pressure is applied, due to the large membrane area to membrane thickness ratio in the cavity structure, the membrane can be dynamically inflated such that the position of electrodes 102 can be adjusted.

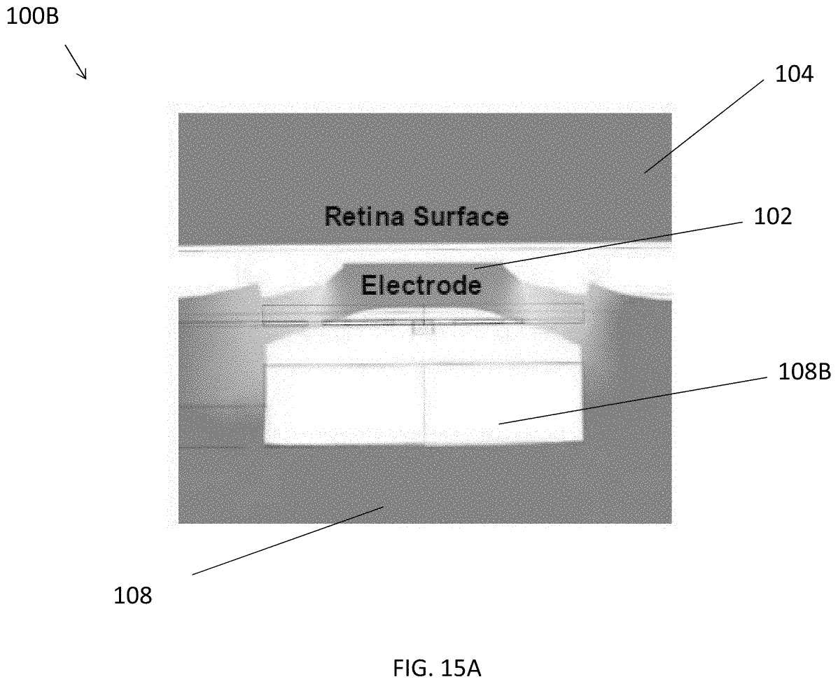

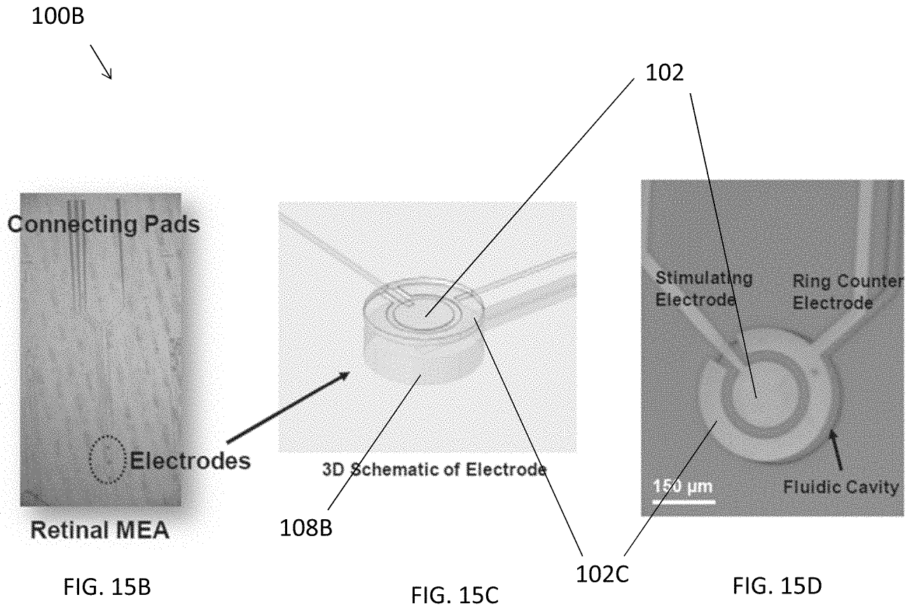

With reference to FIG. 15A, there is shown an illustration of a cross-sectional view of the MEA apparatus 100B similar to FIG. 2B with a single electrode unit 102 with a microfluidic cavity 108B embedded underneath. There is a gap 120 between the electrode 102 and the retina surface 104. By increasing the internal fluidic pressure in the cavity 108B, the electrode 102 may be pushed towards the retina surface 104 to realize a physical contact between the electrode 102 and the retina cell 104, thereby facilitating an electrical signal transmission between such electrode 102 and the contacted retinal cell(s) 104.

By fine adjusting the positions of each of the electrode 102, all the electrodes 102 can form a good physical contact with the target 104, such as the retina cells, even though the substrate of MEA apparatus 100B may not perfectly fit the topography on the retinal surface 104.

Referring to FIGS. 15B to 15D, there is shown two micrographs and a schematic drawing of a portion of the fabricated MEA apparatus 100B and a single electrode unit 102 in the MEA apparatus 100B in accordance with embodiments of the present invention.

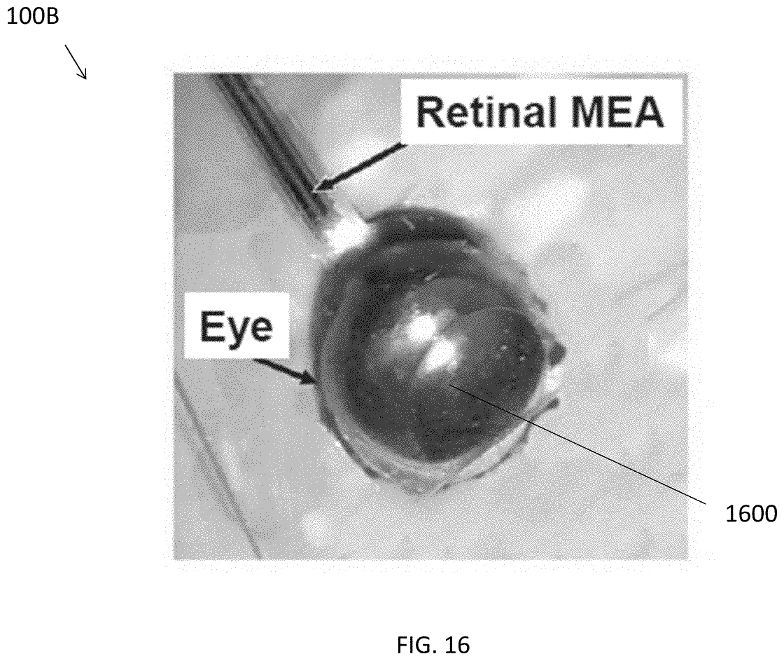

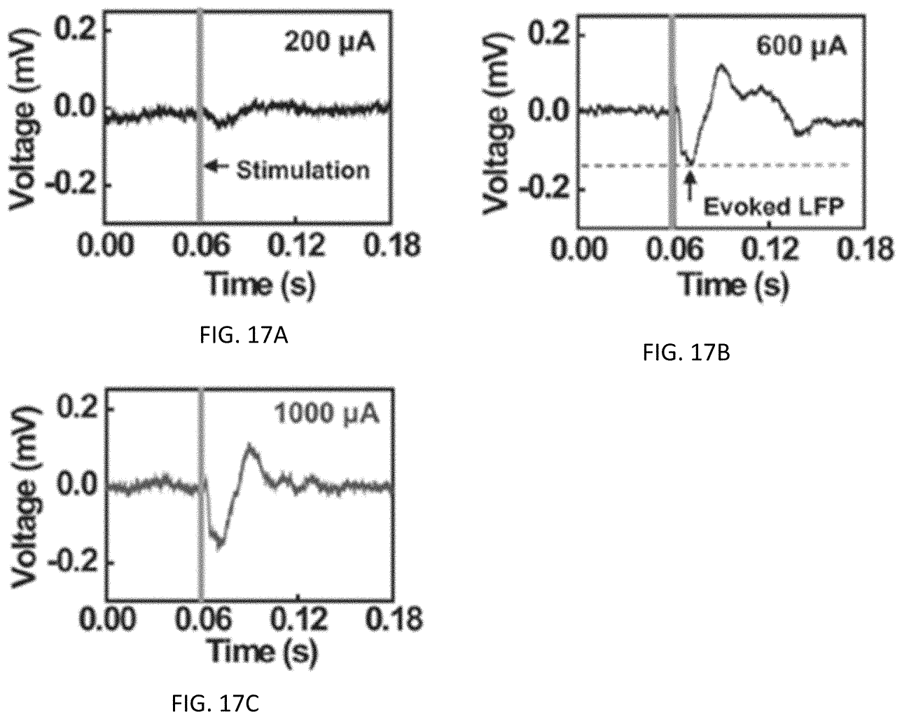

With reference to FIG. 16, there is shown an MEA apparatus 100B implanted in an eye 1600 of an adult male Sprague-Dawley rat, and signal transmission experiments were carried out. Stimulation of retinal cells was achieved by connecting the MEA on to a current source (200 μA, 600 μA, or 1000 μA) which generate current pulses of 0.2 ms duration and 30 s interval. The stimulating signal was delivered to the retina surface, while another neural probe or MEA implanted in the visual cortex in rat brain will pick up the transmitted neural signal. The results are illustrated in FIGS. 17A to 17C.

These embodiments may be advantageous in that the electromedical apparatus such as neural probes and MEA apparatus may be dynamically manipulated with the utilization of embedded microchannels in the apparatus, so as to dynamically control their physical properties.

Advantageously, controlling the stiffness of neural probes may reduce motion induced tissue damage. By adjusting the polymer materials, neural probe dimensions, and fluidic pressure in the microchannels, probe stiffness could be dynamically controlled so that they could be inserted easily into the targeted tissue, but returned to be flexible after implantation by changing the fluidic pressure.

In addition, using the combination of PDMS and PI layers in the developed neural probes with embedded microchannels, the mechanical properties of the probes may be further optimized for deployment and becoming more flexible after penetration to avoid tissue damage.

Besides, the fluidic structure may be fabricated using different materials that allow an adjustment of dimension of it, such that the fluidic structure may be used in micromanipulation of the electrodes in the electromedical apparatus, such as individual electrodes in an MEA apparatus. This may further improve performance of retinal prosthesis or artificial vision apparatus.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. An electromedical apparatus comprising:
an electrical component arranged to facilitate an electrical signal transmission between an electrical device and a target; and
a mechanical component physically connected to the electrical component;
wherein the mechanical component includes a fluidic structure arranged to modify at least one physical property of the mechanical component, so as to facilitate a deployment of the electromedical apparatus on the target.

2. The electromedical apparatus in accordance with claim 1, wherein the target is a biological target.

3. The electromedical apparatus in accordance with claim 1, wherein the fluidic structure comprises at least one fluidic channel arranged to at least temporary accommodate a fluid therein.

4. The electromedical apparatus in accordance with claim 3, wherein the mechanical component comprises a multi-layered structure including at least a first layer of material and a second layer of material.

5. The electromedical apparatus in accordance with claim 4, wherein the at least one fluidic channel is defined by at least one trench formed on the second layer of material and encapsulated by the first layer of material.

6. The electromedical apparatus in accordance with claim 4, wherein the first layer of material and the second layer of material are mechanically flexible.

7. The electromedical apparatus in accordance with claim 4, wherein the first layer of material and the second layer of material are electrically insulating.

8. The electromedical apparatus in accordance with claim 4, wherein the first layer of material and the second layer of material are biocompatible.

9. The electromedical apparatus in accordance with claim 4, wherein the first layer of material includes polyimide.

10. The electromedical apparatus in accordance with claim 4, wherein the second layer of material includes polydimethylsiloxane.

11. The electromedical apparatus in accordance with claim 4, wherein the electrical component includes at least one metal electrode arranged on the first layer of material or the second layer of material.

12. The electromedical apparatus in accordance with claim 3, wherein the at least one physical property includes a mechanical stiffness of the mechanical component, and wherein the mechanical stiffness is modified in response to a variation of an internal pressure of the fluidic channel.

13. The electromedical apparatus in accordance with claim 12, wherein when the electrical apparatus operates in a first condition, the mechanical stiffness of the mechanical component is higher than that of the mechanical component when the electrical apparatus operates in a second condition.

14. The electromedical apparatus in accordance with claim 13, wherein the mechanical component is arranged to prevent bending or buckling of the electromedical apparatus during the deployment of the electromedical apparatus on the target.

15. The electromedical apparatus in accordance with claim 12, wherein the electrical component and the mechanical component combine to define an elongated body, and wherein the fluidic channel runs through at least a portion of the length of the elongated body.

16. The electromedical apparatus in accordance with claim 15, wherein the fluidic structure further comprises an inlet and an outlet both in connection with the fluidic channel.

17. The electromedical apparatus is accordance with claim 16, wherein the inlet and the outlet are define at a first end of the elongated body.

18. The electromedical apparatus in accordance with claim 17, further comprising a tapered tip defined at a second end of the elongated body.

19. The electromedical apparatus in accordance with claim 18, wherein the fluidic channel comprises a U-shaped portion at or proximate to the second end of the elongated body.

20. The electromedical apparatus in accordance with claim 18, wherein the electrical component and the mechanical component combine to operate as a neural probe.

21. The electromedical apparatus in accordance with claim 3, wherein the fluidic structure further comprises at least one fluidic cavity in fluid communication with a respective fluidic channel.

22. The electromedical apparatus in accordance with claim 21, wherein the at least one physical property includes a physical dimension of each of the at least one fluidic cavity, and wherein the physical dimension is modified in response to a variation of an internal pressure in the fluidic channel and/or the fluidic cavity.

23. The electromedical apparatus in accordance with claim 22, wherein, in response to the variation of the internal pressure in the fluidic cavity, the at least one fluidic cavity is arranged to move a respective portion of the electrical component with respect to the target during the deployment of the electromedical apparatus on the target.

24. The electromedical apparatus in accordance with claim 23, wherein the mechanical component is arranged to improve a physical contact between the electrical component and the target.

25. The electromedical apparatus in accordance with claim 24, wherein the electrical component comprises an array of electrodes arranged to electrically contact with the target.

26. The electromedical apparatus in accordance with claim 25, wherein the fluidic structure comprises a plurality of fluidic cavities, wherein the plurality of fluidic cavities defines an array of fluidic cavities.

27. The electromedical apparatus in accordance with claim 26, wherein the size and dimension of the array of electrodes are different from those of the array of fluidic cavities.

28. The electromedical apparatus in accordance with claim 26, wherein each of the electrodes in the electrical component aligns with a corresponding fluidic cavity in the mechanical component.

29. The electromedical apparatus in accordance with claim 25, wherein the electrical component further comprises a counter electrode adjacent to each electrode in the array of electrodes.

30. The electormedical apparatus in accordance with claim 29, wherein each electrode and counter electrode pair is arranged to estimate a position of the electrode with respect to the target so as to evaluate the physical contact between the electrical component and the target.

31. The electromedical apparatus in accordance with claim 25, wherein the electrical component and the mechanical component combine to operate as a micro-electrode array apparatus.

32. The eletrcomedical apparatus in accordance with claim 3, further comprising a pump arranged to manipulate a flow of fluid through the fluidic channel under a predetermine pressure.

33. A method for deploying the electromedical apparatus in accordance with claim 18 on a target, comprising the steps of:
increasing the internal pressure of the fluidic channel;
implanting the neural probe in the target; and
decreasing the internal pressure of the fluidic channel after a disposition of the electrical component in a first predetermined position.

34. The method for deploying the electromedical apparatus on a target in accordance with claim 33, further comprising the step of controlling the internal pressure of the fluidic channel so as to move the electrical component from the first predetermined position to a second predetermined position.

35. A method for deploying the electromedical apparatus in accordance with claim 31 on a target, comprising the steps of:
disposing the micro-electrode array apparatus on the target; and
controlling the internal pressure of each of the plurality of fluidic cavities so as to improve the physical contact between the electrical component and the target at each respective portion of the electrical component.

36. The method for deploying the electromedical apparatus on a target in accordance with claim 35, further comprising the step of estimating a position of each of the electrodes with respect to the target to evaluate the physical contact between each of the electrodes and the target.

37. A method for manufacturing an electromedical apparatus, comprising the steps of:
defining a first layer of material with a fluidic structure; and
combining the first layer of material with a second layer of material to form a multi-layer structure defining a mechanical component with the fluidic structure; and
providing an electrical component to physically connect with the mechanical component, the electrical component is arranged to facilitate an electrical signal transmission between an electrical device and a target;
wherein the fluidic structure is arranged to modify at least one physical property of the mechanical component, so as to facilitate a deployment of the electromedical apparatus on the target.

38. The method for manufacturing an electromedical apparatus in accordance with claim 37, wherein the step of combining the first layer of material with a second layer of material comprises the step of stacking and binding the first layer of material onto a second layer of material.

39. The method for manufacturing an electromedical apparatus in accordance with claim 38, wherein the first layer of material and the second layer of material are combined via chemical bondings.

40. The method for manufacturing an electromedical apparatus in accordance with claim 38, wherein the mechanical component is formed using a reversal imprint process.

41. The method for manufacturing an electromedical apparatus in accordance with claim 38, wherein the step of defining a first layer of material with a fluidic structure comprises the step of applying the first layer of material on a stamp so as to construct the fluidic structure on the first layer of material.

42. The method for manufacturing an electromedical apparatus in accordance with claim 41, wherein the step of defining a first layer of material with a fluidic structure comprises the step of coating a first anti-sticking layer on the stamp to modify a surface energy prior to applying the first layer of material on the stamp.

43. The method for manufacturing an electromedical apparatus in accordance with claim 42, further comprising the step of:
transferring the first layer of material from the stamp to a handling substrate; and
releasing the first layer of material from the handling substrate after binding the first layer of material onto the second layer of material.

44. The method for manufacturing an electromedical apparatus in accordance with claim 43, wherein the step of transferring the first layer of material from the stamp to a handling substrate comprises the step of:
coating a second anti-sticking layer on the handling substrate;
stacking the first layer of material apply on the stamp to the handling substrate; and
releasing the first layer of material from the stamp.

45. The method for manufacturing an electromedical apparatus in accordance with claim 44, wherein the first or the second anti-sticking layer comprise FOTS and/or MOPTS.

46. The method for manufacturing an electromedical apparatus in accordance with claim 37, wherein the step of providing the electrical component comprises the step of fabricating at least one electrode on the first and/or second layer of material.

47. The method for manufacturing an electromedical apparatus in accordance with claim 46, wherein the step of fabricating the at least one electrode on the first and/or the second layer of material comprises the steps of:
depositing a metal layer on the first layer of material and/or the second layer of material; and
patterning the metal layer by photolighography and etching of the metal layer.

48. The method for manufacturing an electromedical apparatus in accordance with claim 47, wherein the at least one electrode is fabricated prior to the step of combining the first layer of material with the second layer of material.

49. The method for manufacturing an electromedical apparatus in accordance with claim 47, further comprising the step of covering the at least one electrode with a passivation layer prior to combining the first layer of material with the second layer material.

50. The method for manufacturing an electromedical apparatus in accordance with claim 37, further comprising the step of exposing at least one electrical contact of the electrical component by etching.

51. The method for manufacturing an electromedical apparatus in accordance with claim 46, wherein the at least one electrode comprises Au and/or Cr.

52. The method for manufacturing an electromedical apparatus in accordance with claim 37, wherein the first layer of material and the second layer of material are biocompatible.

53. The method for manufacturing an electromedical apparatus in accordance with claim 37, wherein the first layer of material and the second layer of material are mechanically flexible.

54. The method for manufacturing an electromedical apparatus in accordance with claim 37, wherein the first layer of material and the second layer of material are electrically insulating.

55. The method for manufacturing an electromedical apparatus in accordance with claim 37, wherein the first layer of material includes polyimide.

56. The method for manufacturing an electromedical apparatus in accordance with claim 37, wherein the second layer of material includes polydimethylsiloxane.

57. The method for manufacturing an electromedical apparatus in accordance with claim 39, wherein the first layer of material and the second layer of material are combined via a layer of binding material sandwiched between the first and the second layers of material.

58. The method for manufacturing an electromedical apparatus in accordance with claim 57, wherein the binding material includes polymethyl methacrylate.

59. The method for manufacturing an electromedical apparatus in accordance with claim 39, wherein the first layer of material and the second layer of material are combined via a layer of adhesive material.

60. The method for manufacturing an electromedical apparatus in accordance with claim 37, wherein the electromedical apparatus operates as a neural probe.

61. The method for manufacturing an electromedical apparatus in accordance with claim 37, wherein the electromedical apparatus operates as a micro-electrode array apparatus.
